(19) United States
(12) Patent Application Publication
(10) Pub. No.: US 2022/0310215 A1
(43) Pub. Date: Sep. 29, 2022

(54) MENTAL HEALTH PLATFORM

(71) Applicant: Bend Health, Inc., Madison, WI (US)

(72) Inventor: Kurt Edward Roots, Madison, WI (US)

(73) Assignee: Bend Health, Inc., Madison, WI (US)

(21) Appl. No.: 17/690,142

(22) Filed: Mar. 9, 2022

Related U.S. Application Data
(60) Provisional application No. 63/167,284, filed on Mar. 29, 2021.

(51) Int. Cl. G16H 10/20; G16H 10/60; G16H 20/00

(52) U.S. Cl. CPC G16H 10/20; G16H 10/60; G16H 20/00

(57) ABSTRACT

The present disclosure describes a medical platform. In some examples, the medical platform can include instructions to generate an electronic health record (EHR) for a user that includes data captured during an intake session and a recommended clinical pathway for the user based on a health score and data captured during a set of questions.

### BACKGROUND

A computing device can allow a user to utilize computing device operations for work, education, gaming, multimedia, and/or other uses. Computing devices can be utilized in a non-portable setting, such as at a desktop, and/or be portable to allow a user to carry or otherwise bring the computing device along while in a mobile setting. These computing devices can be connected to networks that allow remote computing devices to collect data from users.

### BRIEF DESCRIPTION OF THE DRAWINGS

FIG. 1 illustrates an example of a method for executing a mental health platform.

FIG. 2 illustrates an example of a method for executing a mental health platform.

FIG. 3 illustrates an example of a method for executing an evidence-based symptom measure of a mental health platform.

FIG. 4 illustrates an example of a method for executing digital biomarkers of a mental health platform.

FIG. 5 illustrates an example of a computing device for executing a mental health platform.

FIG. 6 illustrates an example of a memory resource storing instructions for executing a mental health platform.

FIG. 7 illustrates an example of a system including a computing device for executing a mental health platform.

### DETAILED DESCRIPTION

A user may utilize a computing device for various purposes, such as for business and/or recreational use. As used herein, the term “computing device” refers to an electronic system having a processor resource and a memory resource. Examples of computing devices can include, for instance, a laptop computer, a notebook computer, a desktop computer, an all-in-one (AIO) computer, networking device (e.g., router, switch, etc.), and/or a mobile device (e.g., a smart phone, tablet, personal digital assistant, smart glasses, a wrist-worn device such as a smart watch, etc.), among other types of computing devices. As used herein, a mobile device refers to devices that are (or can be) carried and/or worn by a user.

Computing devices can utilize network connections to communicate with a plurality of remote computing devices. For example, a first computing device can utilize a network connection (e.g., local area network (LAN), wide area network (WAN), Internet, etc.) to transfer and receive information from a plurality of remote computing devices. In some examples, the computing devices can utilize remote resources through network connections with server devices or cloud resources. Computing devices and communication networks can provide added functionality to the field of medicine.

Telehealth companies have sought to solve access and quality issues in the field of medicine, but are constrained by experts to staff their delivery models to meet growing demand and shrinking expert supply. Digital tools and the utilization of lower cost staff has helped shoulder the burden of mental health staffing shortages, but it has fallen dramatically short of meeting the demands for mental health care. This problem will increase dramatically within the next ten years when 55% of the total psychiatrist workforce is expected to retire.

To deal with the shortage of mental health experts that can prescribe, significant efforts over the last decade have been made to integrate behavioral health into primary care. Despite the intuitive appeal of getting primary care physicians to provide more evidence-based mental health care, making this happen faces formidable challenges. Primary care physicians vary widely in their interest, commitment, confidence, and, most importantly, skill and effectiveness in evaluating and treating mental health problems.

The lack of access to expert prescribers exacerbates the quality problems at the primary care level. Therefore, to solve this problem, the present platform optimizes the scarce resource of mental health experts through evidence-based clinical decision support and empowers primary care physicians with this clinical support. This is done because the primary care physicians will remain the primary home for patient engagement. The present disclosure describes a platform that includes a plurality of functions to generate electronic health records (EHRs) utilizing data collected through a plurality of dynamically generated questions, a plurality of screening tools, and historical data associated with a plurality of different recommended clinical pathways for a plurality of remote patients or users of the platform.

Because of the shortage of mental health experts, this solution optimizes their work instead of replacing them with lower cost, more accessible providers. The latter solution is a common approach taken today which leads to poor outcomes and limits the scope of conditions and treatments available. This invention optimizes this work through three approaches. In some examples, the system uses adaptative intake and follow-up methodology to objectively measure symptoms and acuity. This system recommends clinical pathways for treatment and evaluates it over time. In these examples, treatment is initiated and delivered through a purpose-built EHR that contains clinical workflows and shortcuts for delivering specialized care.

Traditionally, the heterogeneity in the clinical presentation of mental health disorders and response to therapy and/or medication limits clinicians' ability to accurately predict a specific patient's eventual response to treatment. The high variability of symptom presentations and associated clinical trajectories present formidable challenges for clinician decision making. As a consequence, typical treatment selection for mental health conditions occurs on a “try-and-try-again” basis, based on lack of perceived treatment benefit by patients and clinicians. There is therefore a significant need to derive accurate and quantitatively-based prognoses of eventual treatment outcomes, given a set of measured changes in symptom severity and other factors at specific intervals, before therapeutic trials are declared to be fully complete.

The present disclosure describes a platform for solving the mental health access and quality problem through a consumer-centric and data-driven healthcare service leveraging AI, care teams, and expert psychiatrists to evaluate and provide treatment options such as medications and therapy to improve mental health outcomes. The platform can utilize a wide assortment of data based on behavioral, biological and genetics data and the platform can provide treatment recommendation and specific clinical decision support through AI to care teams and clinical experts. Through the AI-based recommendations, this platform provides an empirical platform across consumers, mental health disorders, and treatment options to accurately identify the optimal treatment plan leveraging medication(s) and/or therapy, creating symptom trajectory improvements across time through accurately predicting remission, response, or nonresponse. Reducing time to treatment reduces treatment costs, improves outcomes, and reduces time spent by the psychiatrist and other practitioners in the system.

The platform comprises a plurality of continuous data inputs. Some examples of inputs provided by users into the system include data inputs related to mental health conditions, multimedia inputs related to facial expressions, emotions and cues, data inputs related to calculations and metrics related to human characteristics, including but not limited to exercise data, heart rate variability (HRV), sleep data and oxygen levels, and data inputs including but not limited to genetic sequence, expression, and variant data.

The platform also has the ability to transform these various types of raw data inputs into usable features for statistical and probabilistic models. The models (e.g., AI-models, machine learning models, etc.) comprise statistical and probabilistic functions which can prospectively identify multiple treatment outcomes across therapy and medication options. In addition to these examples of capabilities of the platform, it also utilizes sophisticated feedback loops, which as an example, can represent a plurality of technologies including prompts, inputs, SMS, fax, messaging, email and phone support to facilitate continuous data capture from consumers and engagement between providers and primary care physicians.

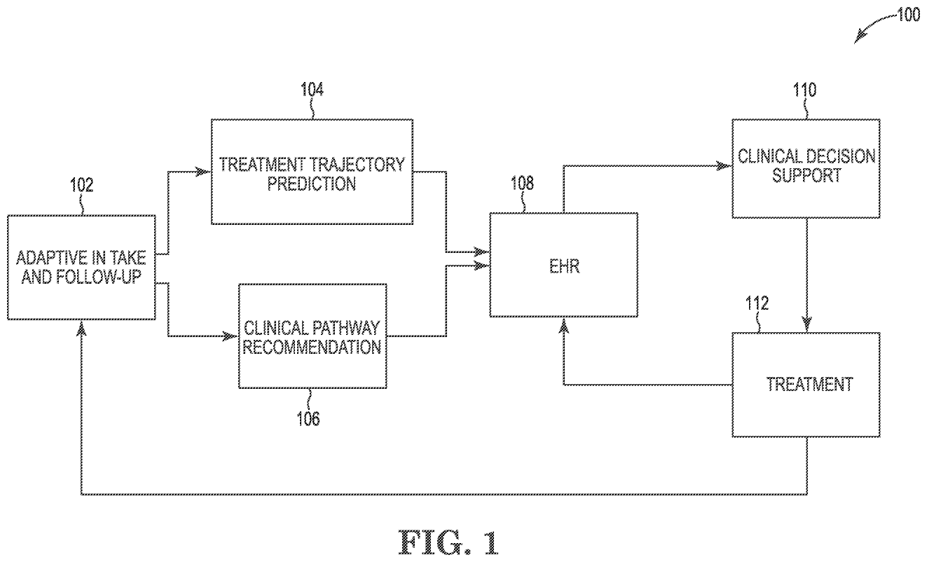

FIG. 1 illustrates an example of a method 100 for executing a mental health platform. As used herein, the mental health platform is a digital environment that allows a plurality of users to execute functions of the platform. In some examples, a plurality of application programming interfaces are utilized to execute the functions described herein for a plurality of different users.

The method 100 illustrates a plurality of steps that can be performed or executed by a computing system running the mental health platform. As used herein, a computing system includes a plurality of computing devices that are communicatively coupled to perform particular functions. The method 100 can be utilized by a plurality of different users including, but not limited to: patients, primary care providers, health coaches, nurses, clerks, scribes, executive assistances, among other users that can be part of providing or assisting in treatment 112 for a particular patient.

In some examples, the method 100 can be utilized by the computing system to generate an electronic health record (EHR) 108 for a plurality of patients or users of the method 100. In some examples, the method 100 can begin with an adaptive intake and follow-up at 102. As described further herein, the adaptive intake and follow-up at 102 can include collecting a plurality of quantitative and qualitative data related to a particular user (e.g., patient). In some examples, the adaptive intake and follow-up at 102 includes collecting digital biomarkers, collecting evidence-based system measures, and collecting patient entered data.

As described further herein, collecting the patient entered data can include collecting inputs through a remote computing device related to the health history of the patient. In some examples, the patient entered data of the patient includes, but is not limited to: medical history, mental health history, medication history, demographics, pharmacy, primary care physician, education, biological data, genetic data, or other data related to a current health of the patient. In some examples, wearable devices can be used to monitor heart rate, a quantity of sleep, resting heart rate, quantity of calories burned, among other health related data that can be captured by a wearable device.

Collecting the evidence-based system measures includes utilizing a plurality of platform screening tests that are executed to test for a plurality of different conditions. For example, each of the plurality of screening tests include a set of corresponding questions that are used to generate a score for the patient. The score can be utilized to indicate a likelihood of a corresponding condition. As described further herein, the plurality of platform screening tests can be designated based on the age of the patient and the score can be based on the responses provided by the patient. In some examples, the mental health platform is able to identify a particular condition is more probable during the execution of the plurality of screening tests and dynamically expands the set of questions associated with the particular condition. For example, the mental health platform can algorithmically expand the set of questions for a particular health condition based on answers to particular questions of the screening test for the particular health condition.

Collecting the digital biomarkers can include collecting audio and video data of a patient's response to questions generated by the mental health platform. As described further herein, the mental health platform can generate questions that are provided to a patient. The patient is then instructed to respond to the generated questions and the responses can be captured utilizing imaging devices (e.g., camera, video camera, etc.) to capture video data of the responses and microphone devices to capture audio data of the responses. As described further herein, the audio data and video data can be utilized to extract features of the responses that can be utilized generate follow-up questions for the patient and/or provide additional data for generating a clinical pathway recommendation at 106.

The method 100 includes generating a clinical pathway recommendation at 106. The clinical pathway recommendation includes a plurality of tasks to be performed over a particular treatment. For example, the plurality of tasks to complete can include initiating contacts between the patient and a team of medical professionals to complete the treatment process, verifying insurance, collecting payment information, setting up calendar appointments with a psychiatrist or other providers, setting up calendar appointments with mental health coaches, among other tasks associated with completing the treatment. As described further herein, the plurality of tasks can be associated or assigned to a plurality of different users associated with the treatment process. For this reason, the clinical pathway recommendation can include a checklist of the plurality of tasks that can be confirmed by a corresponding user when completed and a different user of a subsequent task can be notified that the task was completed.

In some examples, the method 100 includes generating a treatment trajectory prediction at 104. The treatment trajectory prediction can be an estimation of success of a treatment based on the medical information of the patient. For example, the treatment trajectory prediction can be a probability curve of success based on historical patterns associated with the treatment for similar patients based on a plurality of health information associated with the patient. For example, the model for the treatment trajectory prediction can utilize age, gender, primary condition, ethnicity, assessment scores, medical history, medication history, genetic profile, treatment plan, digital biomarkers, among other health information that can be associated with efficacy for the treatment plan of the patient. In some examples, treatment predictions (e.g., treatment trajectories) can inform patients and their treatment team about efficacy of treatment relative to these (age, gender, condition, treatment, etc.) factors, and allows for adjustments to be made earlier in the course of treatment, instead of waiting months or years.

In some examples, the data collected during the adaptive intake and follow-up at 102, the treatment trajectory prediction at 104, and/or the clinical pathway recommendation at 106 can be stored as an electronic health record (EHR) at 108. The electronic health record can be a secure database that allows a patient to interact with the adaptive intake and follow-up at 102 without having access to health-related information of other patients. The electronic health record can also be utilized to allow a plurality of medical professionals and support staff to limited access to patient related information based on user credentials. For example, a particular medical professional may have access to a first user's medical records but not a second user's medical-records. In a similar way, the medical professional may have access to a first user's medical records while a support staff may have limited access to the first user's information (e.g., address information, contact information, billing information, etc.) for performing a particular task.

The method 100 includes providing information from the electronic health record to provide clinical decision support at 110. The clinical decision support can include providing health information to a medical professional to enable the medical professional to provide a recommendation with the information from the electronic health record. In this way, the medical health professional can make a diagnosis or treatment decision with relatively less time.

The method 100 includes logging the treatment at 112. The treatment information that is logged or collected at 112 can be provided to the electronic health record at 108 and provided to the adaptive intake and follow-up at 102. Specifically, the collected treatment data 112 can be utilized to generate additional questions or follow-up questions to a particular patient at 102. In some examples, the collected treatment data at 112 can allow the method 100 to monitor the progress of a particular patient and utilize the progress to alter the clinical pathway recommendation and/or update the treatment trajectory projection for the particular patient.

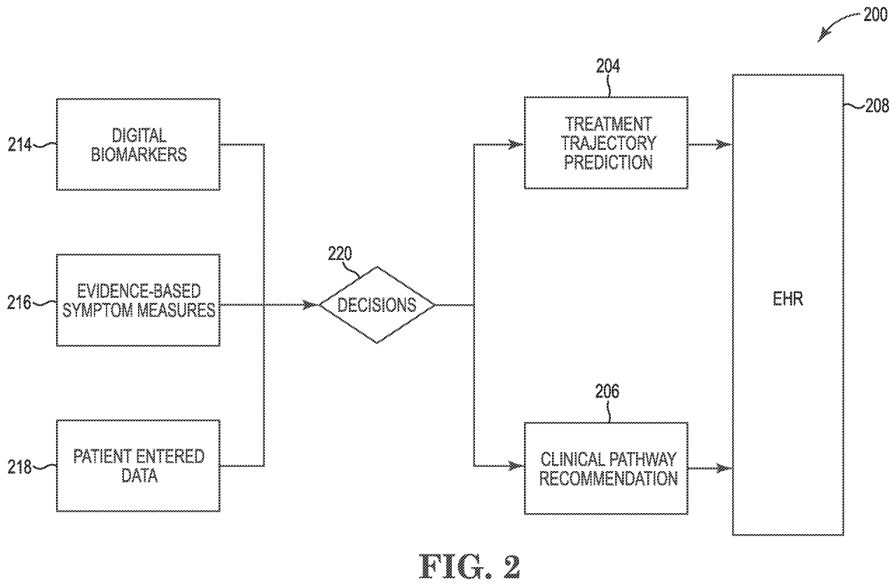

FIG. 2 illustrates an example of a method 200 for executing a mental health platform. In some examples, the method 200 is a portion of the method 100 as illustrated in FIG. 1. For example, the method 200 illustrates a portion of the method 100 to generate an electronic health record (EHR) 208. In this example, the adaptive intake and follow-up at 102 of the method 100 is broken into collecting digital biomarkers at 214, collecting evidence-based symptom measures at 216, and collecting patient entered data at 218. In some examples, the data is collected through a user interface generated on a display of a remote computing device provided to a user or patient. The data that is collected can be utilized to generate a decision at 220.

The adaptive intake and follow-up of the method 200 includes collecting a variety of data related to a particular patient at an initial intake session as well as at regular or somewhat regular follow-up sessions. The patient entered data can provide a broad description and a detailed description of a health history of a particular patient. In some examples, the health history of a particular patient can be utilized to generate the clinical pathway recommendation at 206 and/or be utilized to predict the treatment trajectory prediction at 204. In some examples, collecting the patient entered data at 218 includes providing a user interface through a display device to allow a patient to enter data including, but not limited to: medical history data, mental health history data, medication history data, demographics data, pharmacy history data, primary care physician information, school data, biological data, genetic data, and/or other types of data. In some examples, the user interface can be utilized to upload medical documents that can include the information to be provided for the patient.

In other examples, the user interface can be utilized to provide access to a remote computing device to access a patient's medical records from primary care providers or other medical providers. For example, the user interface can display agreement documents for allowing an organization associated with the mental health platform to access a particular patient's medical records associated with different medical professionals. In other examples, the user interface can allow the user to link accounts with the mental health platform of method 200. For example, the patient may have genetic data collected by a third-party organization. In this example, the patient can utilize the user interface to link information of the genetic data from the third-party organization to the mental health platform.

In some examples, the user interface can allow a plurality of peripheral devices to collect samples of biological data. For example, the biological data can include, but is not limited to: heartrate, heart rate variability (HRV), blood pressure, height, weight, temperature, among other data related to a user's current health condition. In some examples, the plurality of peripheral devices can include a blood pressure cuff and/or heart rate monitor to provide the biological data to the mental health platform. The data collected by the plurality of peripheral devices can be utilized during a decision at 220 in view of data collected by evidence-based symptoms measures at 216 and/or collected digital biomarkers at 214.

In some examples, the method 200 includes collecting evidence-based symptom measures at 216. Collecting evidence-based symptom measures includes utilizing screening measures for mental health conditions. As used herein, screening measures are a set of questions that include multiple choice answers that are scored to determine whether a particular health condition may be present for a particular patient. In some examples, a screening measure can be a standardized test to screen for a specific health condition. For example, a first screening measure can be a first set of questions that is specifically calibrated for a particular age group to determine whether a patient is likely to suffer from anxiety while a second screening measure can be a second set of questions that is specifically calibrated for a particular age group to determine whether a patient is likely to suffer from attention-deficit/hyperactivity disorder (ADHD).

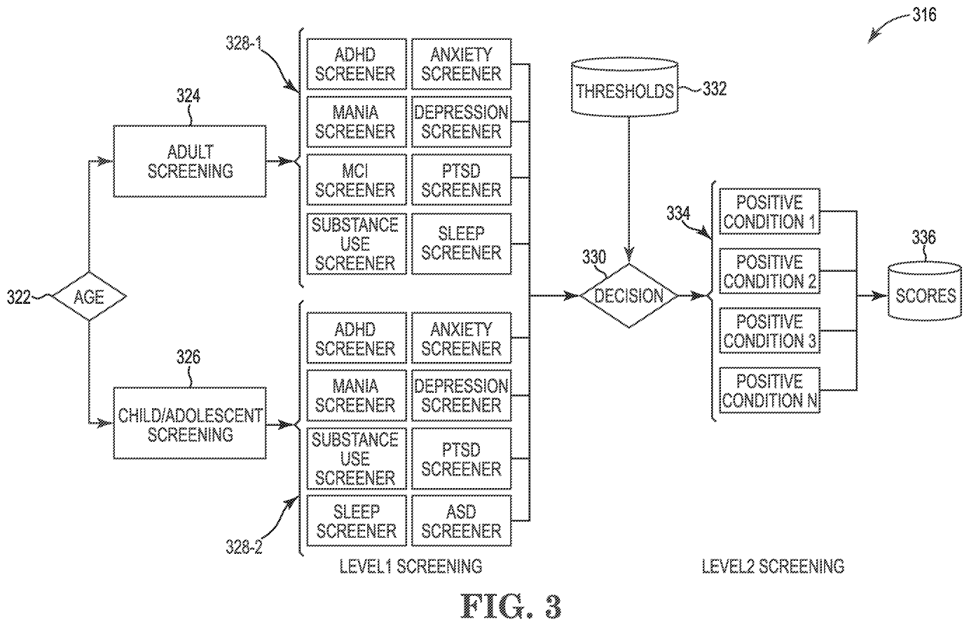

The screening measures that can be utilized by the mental health platform include, but are not limited to: Patient Health Questionnaire-9 (PHQ9), General Anxiety Disorder-7 (GAD7), Patient-Reported Outcomes Measurement (PROMIS), Montreal Cognitive Assessment (MoCA), Swanson, Nolan, and Pelham Rating Scale (SNAP-IV), Y-BOCS, among other screening measures that can be utilized to identify a patient with a particular medical condition (e.g., anxiety, depression, ADHD, sleep deprivation, substance abuse, mania, trauma, etc.). In some examples, a plurality of screening measures can be built into the mental health platform as a dynamic decision tree. In this way, the patient can be tested for a plurality of health conditions by answering generated questions through the user interface. The screening measures and method for executing the evidence-based symptom measures at 216 is further described in reference to FIG. 3.

In some examples, the method 200 includes collecting digital biomarkers at 214. Digital biomarkers include biological markers that are identified from digital data such as video data and audio data. For example, digital biomarkers from audio data can include, but are not limited to: tone, pitch, volume, among other features. In some examples, collecting digital biomarkers includes capturing audio and/or video data of a patient reading a script or answering relatively easy questions. The captured audio and/or video data of a patient reading a predetermined script or answering relatively simple questions can be utilized to generate a biomarker baseline for the particular patient that can be utilized to compare to audio and/or video data of the patient answering medical related questions.

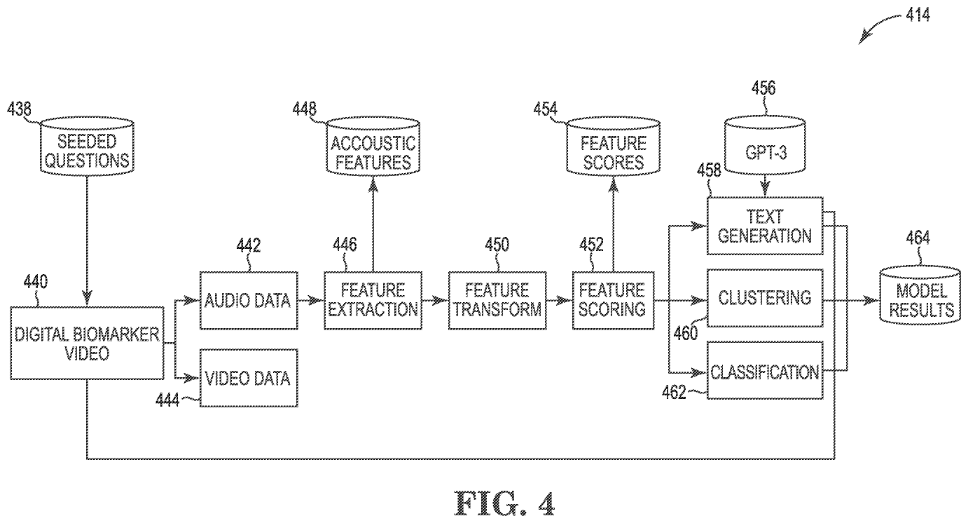

In other examples, the biomarker baseline for the particular patient can be compared to subsequently captured audio and/or video data of the particular patient reading the predetermined script. In this way, the comparison between interactions with the user interface of the mental health platform can be utilized to monitor progress of the patient over time. In addition, the biomarker baseline for each session can be utilized as the baseline for the corresponding session. Capturing the digital biomarkers at 214 is further described with reference to FIG. 4.

The method 200 includes making a decision at 220. In some examples, the decisions at 220 are based in part on the collected digital biomarkers at 214, the evidence-based symptom measures at 216, and/or the patient entered data at 218. In some examples, the decisions at 220 can include identifying potential medical conditions of the patient, severity of the potential medical conditions of the patient, scoring associated with the plurality of evidence-based symptom measures at 216, subsequent questions to generate and provide to the patient, among other decisions or generated data that can be stored in the secure electronic health record at 208.

As described herein, the method 200 can also include generating a clinical pathway recommendation at 206 and a treatment trajectory prediction at 204 that can be stored in a secure electronic health record at 208. As described herein, the clinical pathway recommendation can include a plurality of tasks to be performed by the medical team of the patient and/or a plurality of tasks to be performed by the patient. In these examples, the plurality of tasks can be assigned to particular users (e.g., medical professional, patient, administration, etc.) such that all of the plurality of tasks are performed subsequent to pre-required tasks. For example, a task of assigning a medical professional would be a pre-required task to setting up a consultation between the patient and the medical professional. In this way, the clinical pathway recommendation can be utilized to organize the interactions between a plurality of different users of the mental health platform to execute the treatment plan.

FIG. 3 illustrates an example of a method 316 for executing an evidence-based symptom measure of a mental health platform. In some examples, the method 316 can further illustrate the method of evidence-based measures at 216 of method 200 as illustrated in FIG. 2. As described herein, the method 316 can illustrate instructions that are executed by a computing device or computing system that is executing the mental health platform.

The method 316 includes determining an age of a patient or user of the mental health platform at 322. In some examples, determining the age of the patient or user can be utilized to categorize the patient or user as an “adult” or “child”. As described herein, the mental health platform can utilize a plurality of screening measures 328-1, 328-2 to determine a potential condition of the patient or user of the mental health platform. In these examples, the plurality of screening measures 328-1, 328-2 can be configured to be utilized by a particular age range. In this way, the plurality of screening measures 328-1, 328-2 can be separated into an adult range and a child/adolescent range. For example, the plurality of screening measures 328-1 can be configured to be utilized for adults and the plurality of screening measures 328-2 can be configured to be utilized for children or adolescents.

In this way, the age of the patient is determined at 322 and the patient is provided with the adult screening at 324 if it is determined the patient is within the adult age range and the patient is provided with the child/adolescent screening at 326 if it is determined the patient is within the child/adolescent age range. In some examples, the adult screening at 324 includes an adult set of screening measures 328-1 that can each include a plurality of calibrated questions that can be utilized to score an adult patient's likelihood of having a particular medical condition. As described herein, the adult set of screening measures 328-1 can be utilized to generate questions utilizing a dynamic decision tree to present questions to the patient or user over a period of time. In a similar way, the child/adolescent screening at 326 can include a child/adolescent set of screening measures 328-2 that include calibrated questions for an age range associated with a child or adolescent to generate a score for a child or adolescent patient's likelihood of having a particular medical condition.

As described herein, answers provided by the patient can be utilized to dynamically expand or contract questions related to different conditions. For example, questions for a first screening measure can be expanded or provided to the patient while questions for a second screening measure can be contracted or not provided to the patient based on previous answers provided by the patient. In this way, the answers provided by the patient can allow the mental health platform to provide more detailed questions for particular screening measures associated with particular conditions while providing less questions for other screening measures associated with other conditions.

In some examples, the answers to the plurality of questions provided by the user can be utilized to generate decisions at 330. In some examples, the decisions at 330 are based on a plurality of threshold 332 associated with the screening measures 328-1, 328-2. For example, responses provided by a user to the plurality of questions associated with the screening measures 328-1, 328-2 can be compared to the plurality of thresholds 332 to identify a quantity of positive conditions 334. In some examples, each of the plurality of thresholds 332 corresponds to a particular screening measure of the plurality of screening measures 328-1, 328-2. In some examples, a positive condition of the plurality of positive conditions can be identified when a score is calculated based on answers to a corresponding screening measure for the positive condition exceeds a threshold value of the plurality of thresholds 332.

In some examples, both the actual response score from the patient and the threshold score are captured, which is known as a priori. The actual response score and threshold score are stored as a numeric variable, and both the actual score and the threshold are assigned to each question of the plurality of screening measures 328-1, 328-2. The thresholds 332 are defined by the rules of the plurality of screening measures 328-1, 328-2. In some examples, the mental health platform can generate a message (e.g., SMS, email, chat session, etc.) that is sent to the patient on a regular basis (e.g., monthly, weekly, etc.). The mental health platform digitally collects the numeric responses for the patient for each initial screening question. These scores are stored individually for each question but are also aggregated in a plurality of dimensions. For example, they're aggregated and computed by mental health dimensions (which is a collection of questions relating to a domain like depression, anxiety, mania, etc.), for just one question, and for all questions in the plurality of screening measures 328-1, 328-2. This allows for multiple types of granular comparisons over time.

In some examples, the mental health platform compares, in real-time, the score of the responses (e.g., numeric responses) to the threshold value. If the score of the response is greater than or equal to the threshold, the mental health platform can trigger additional screening for the corresponding condition associated with the questions. In these examples, the mental health platform iteratively applies the same scoring and the threshold triggering logic to the new questions and responses to produce a complete and comprehensive quantitative picture of the patient that can be measured longitudinally. The mental health platform also automatically calculates the overall assessment scores for a patient and determines whether the patient screens positively for one or more conditions, along with severity of the one or more conditions (e.g., positive conditions 334).

In some examples, the plurality of positive conditions 334 can be utilized to generate additional questions that are presented to the user or patient. For example, the user can be presented with a first plurality of questions associated with the screening measures 328-1, 328-2 and the presented with a second plurality of questions associated with the plurality of positive conditions 334. In these examples, the additional questions can be utilized to generate a score for the plurality of positive conditions 334 to determine if one or more of the positive conditions 334 are likely to be a diagnosis for the patient and/or should be considered for a treatment associated with the patient. In these examples, the plurality of scores 336 can be stored in the electronic medical record associated with the patient or user.

In this way, the plurality of scores 336 can be utilized to identify a diagnosis of a particular condition for the patient, severity of a particular condition of the patient, a clinical pathway recommendation for the patient, and/or the treatment trajectory prediction for the patient. In some examples, the plurality of scores 336 can be compared to a plurality of other scores associated with other users to identify clinical pathway recommendations based on outcomes associated with the users that received the other scores. In some examples, the plurality of scores 336 can be compared to previous or subsequent scores associated with the particular patient to identify the progress of a particular treatment and/or update the treatment trajectory prediction for the patient. In this way, the treatment or clinical pathway recommendation can be updated more rapidly compared to previous methods.

FIG. 4 illustrates an example of a method 414 for executing digital biomarkers of a mental health platform. In some examples, the method 414 further illustrates collecting digital biomarkers at 214 of method 200 as illustrated in FIG. 2. For example, the method 414 can be executed by a computing device for collecting digital biomarkers of a user answering questions provided by the mental health platform. As described herein, the digital biomarkers can be extracted from audio and/or video data captured from the patient and/or guardian of the patient during responses to questions provided by the mental health platform.

The digital audio biomarkers can include, but are not limited to: measures of pitch, intonation, vocal formats, fundamental frequency, loudness, and inter-word pause length, among other audio biomarkers. The digital video biomarkers can include, but are not limited to: facial expressions, eye direction, facial muscle position, among other facial features or expressions that can correspond to particular behaviors. In some examples, the digital biomarkers can include a combination of audio biomarkers and video biomarkers that are captured during the answers to questions generated by the mental health platform.

In some examples, the method 414 includes generated seeded questions 438 that can be provided to the user interface provided to the user. In some examples, the seeded questions can be based on an age of the patient and/or the screening measures from the intake session. The user is able to record an answer to the seeded questions to capture the digital biomarker video at 440. In some examples, the captured digital biomarker video at 440 can be separated into audio data 442 and video data 444. Separating the audio data 442 and video data 444 can allow feature extraction of the audio data at 446. In some examples, the features of the audio data 442 that are extracted include the audio biomarkers. In some examples, the audio features of the audio data 446 extracted at 446 are compared to a database of acoustic features 448.

In some examples, the comparison between the extracted features of the audio data at 446 and the database of acoustic features 448 can identify a classification of the extracted features of the audio data at 446. The different classifications of extracted features can be utilized to perform a feature transform at 450. In some examples, a feature transform can include transforming the audio feature from an acoustic signal to a digital signal. In some examples, a Fourier transform (FT) can be utilized on the features that were classified based on the comparison. As used herein, a Fourier transform is a mathematical transform that decomposes functions depending on space or time into functions depending on spatial or temporal frequency, such as the expression of a vocal pattern in terms of the volumes and frequencies.

In some examples, the method 414 includes generating a feature score at 452. In these examples, the plurality of features that are extracted from an answer to a particular question can be scored based on relevance to particular medical conditions. In some examples, the plurality of features are scored based on a plurality of factors associated with extracted features. For example, the plurality of features can be utilized to generate quantitative models of the patient and/or guardians (e.g., parent, caregiver, legal guardian, etc.) of the patient. In some examples, the quantitative models can be based on measures of pitch, intonation, vocal formats, fundamental frequency, loudness, and/or inter-word pause length.

In some examples, a script can be utilized to generate a baseline for a particular patient and the baseline can be utilized to score the plurality of features for the particular patient. In these examples, the score can be based on a difference between the generated baseline for the particular patient and a particular response for the plurality of measures of the plurality of features. For example, the differences in pitch, intonation, vocal formats, fundamental frequency, loudness, and/or inter-word pause length can be calculated between a generated baseline for the patient and/or guardian and an answer to a particular question. In some examples, the baseline is generated to calibrate and quantitively model the patient's vocal patterns. By standardizing this ingestion and performing it regularly (e.g., monthly, weekly, etc.), the platform can more accurately model changes in these acoustic features over time. In these examples, the mental health platform produces both standardized baseline text and dynamically generated text to elicit responses from patients and their family and/or caregivers.

In some examples, a score of an answer can be based on a difference between extracted features during a supervised and unsupervised answer to a particular question. For example, a patient or user of the mental health platform can be a minor that may utilize a guardian, parent, or other supervisor during the intake session or while answering particular questions. In this way, a baseline may be generated for a patient, a guardian, the patient when supervised by the guardian, and/or the patient when not supervised by the guardian, among other scenarios. In this way, the extracted audio features of the patient may be compared between questions when the patient is supervised and when the patient is not supervised.

In some examples, the features scores are stored in a feature score database 454. In some examples, the feature score database 454 can be utilized to generate models for a plurality of users based on feature scores of responses provided by the plurality of users over time. In some examples, the features scores are utilized to generate a subsequent question to the answer that was received and utilized to generate the feature score. In one example, a seeded question from the seeded question database 438 is provided to a user and the response to the seeded question is scored and utilized to generate a subsequent question based on the scoring. In another example, a generated question based on a previous response is provided to the user and an answer to the generated question is scored and utilized to generate a subsequent question based on the scoring.

In some examples, the feature scoring at 452 includes generating qualitative modeling of the plurality of features or classification at 462. As described herein, the plurality of features that are extracted from a user's answer to a particular question can be put into a particular classification and/or utilized to generate a model to be stored at model results database 464. For example, the qualitative modeling can include, but is not limited to: emotion classification, sentiment classification, mood classification, condition classification, condition improvement, and/or other qualitative modeling. In this way, the mood or emotion can be identified during a particular response to a generated question and a subsequent question can be generated based on the emotional response to the question.

In some examples, the feature scoring is utilized for clustering the plurality of extracted features. Clustering the plurality of extracted features can be utilized to categorize or generate clusters of feature scores with similar feature scores of other patients or known score ranges for particular conditions. This clustering or categorization can be stored in the model results database 464.

In some examples, a subsequent question can be generated utilizing an autoregressive language model such as GPT-3 resource 456 that can utilize deep learning to generate text for text generation at 458. In this way, the subsequent questions can be generated in a human-like way and provided to the user. The generated question can be sent back to be displayed to the user and/or provided in audio form to the user and responses can be captured utilizing the digital biomarker video at 440. When additional responses are collected, the method 414 can continue to generate subsequent questions that are tailored to collect additional information from the user based on the particular identified conditions of the user.

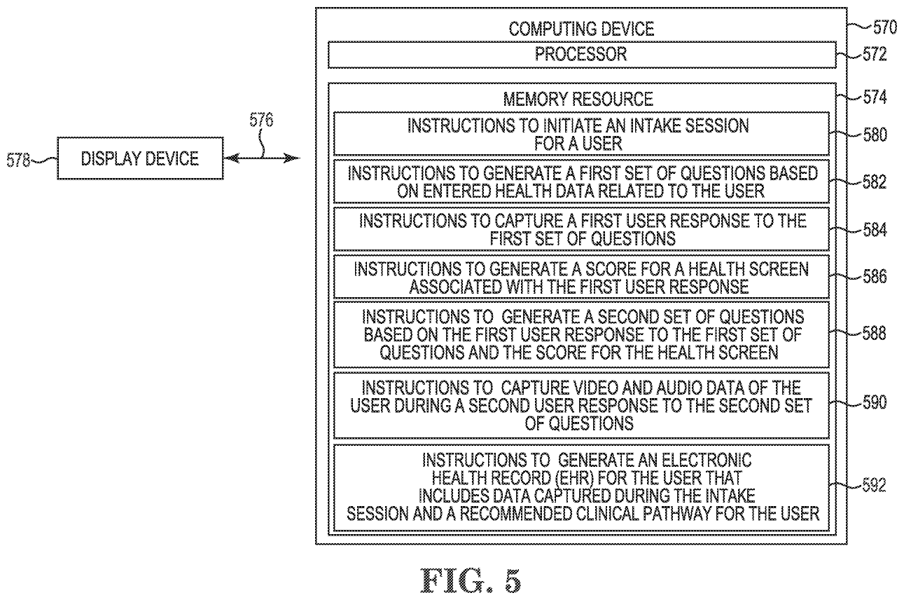

FIG. 5 illustrates an example of a computing device 570 for executing a mental health platform. The computing device 570 can be a device that can execute the methods described herein. In some examples, the computing device 570 can be one of a plurality of computing devices that are utilized in a computing system to perform the methods of executing the mental health platform described herein. For example, the computing device 570 can be a computing server that can communicate with a plurality of computing devices through a communication path or network connection.

In some examples the computing device 570 can include a processor resource 572 communicatively coupled to a memory resource 574. As described further herein, the memory resource 574 can include instructions 580, 582, 584, 586, 588, 590, 592 that can be executed by the processor resource 572 to perform particular functions. In some examples, the computing device 570 is coupled to a display device 578. The display device 578 can be communicatively coupled to the computing device 570 by a communication path 576. The communication path 576 can be a wired or wireless connection to allow the computing device 570 to communicate with the display device 578. In some examples, the computing device 570 can be a remote computing device such as a server device or cloud resource that is communicatively coupled to the display device 578 through a network connection.

The computing device 570 can include components such as a processor resource 572. As used herein, the processor resource 572 can include, but is not limited to: a central processing unit (CPU), an application specific integrated circuit (ASIC), a field programmable gate array (FPGA), a metal-programmable cell array (MPCA), a semiconductor-based microprocessor, or other combination of circuitry and/or logic to orchestrate execution of instructions 580, 582, 584, 586, 588, 590, 592. In other examples, the computing device 570 can include instructions 580, 582, 584, 586, 588, 590, 592, stored on a machine-readable medium (e.g., memory resource 574, non-transitory computer-readable medium, etc.) and executable by a processor resource 572. In a specific example, the computing device 570 utilizes a non-transitory computer-readable medium storing instructions 580, 582, 584, 586, 588, 590, 592, that, when executed, cause the processor resource 572 to perform corresponding functions.

The computing device 570 can be utilized to execute the mental health platform and perform a plurality of methods as described herein. The computing device 570 can be utilized to set up permissions for a plurality of users and generate corresponding user interfaces to allow the plurality of different users to interact with the mental health platform within the permissions set by the computing device 570. In some examples, the computing device 570 can utilize a plurality of screening measures to identify potential conditions of a particular user, generate subsequent questions based on answers to previous questions, and generate a recommended clinical pathway for the particular user that allows a plurality of medical professionals to be notified of tasks associated with the recommended clinical pathway. In this way, the computing device 570 can execute a mental health platform that is capable of providing mental health services for patients from an initial intake through a treatment plan.

In some examples, the computing device 570 can include instructions 580 that can be executed by a processor resource 572 to initiate an intake session for a user. In some examples, the intake session is an interaction with a user interface of the mental health platform. In some examples, the intake session is an initial intake session for a particular user or particular patient. As used herein, an initial intake session for a particular patient is a first interaction for the particular patient with the user interface of the mental health platform.

The intake session can be initiated by requesting authentication information from a user. For example, requesting authentication information can include requesting a username and password combination to access the mental health platform. As described herein, the mental health platform can be utilized by a plurality of users that each have different access credentials. For example, a first user can be a patient that has access to uploading medical information, answering questions generated by the mental health platform, and/or receiving updates from the mental health platform regarding the patient's medical information. In this example, a second user may be a medical provider that has access to the medical information of the first user when the first user is a patient of the medical provider. The different access credentials allow for different access to information stored by the mental health platform, which can be important for complying with different medical regulations (e.g., Health Insurance Portability and Accountability Act of 1996 (HIPAA), etc.).

In some examples, the access credentials can be utilized by the mental health platform to determine access to data stored by the mental health platform and/or determine a portion of users that are to receive particular notifications. For example, notifications can include information protected by different medical regulations and therefore need to be generated and provided to individuals that are in compliance with the medical regulations. In some examples, the user interface can include different functions based on the access credentials of the user. For example, particular tabs or information may be displayed for different users based on the access credentials.

In some examples, the computing device 570 can include instructions 582 that can be executed by a processor resource 572 to generate a first set of questions based on entered health data related to the user. In some examples, the first set of questions can be seeded questions that are based on a user's (e.g., patient's, etc.) entered medical data. As described herein, a patient can enter medical related data into the user interface of the mental health platform. The patient entered data can include, but is not limited to: age, sex, weight, height, illness history, medication history, among other medical information that can be deemed relevant to particular health conditions.

In some examples, the first set of questions can be screening measures (e.g., screening tests, etc.) that can be administered to the user based on the patient entered health data. In some examples, the mental health platform can utilize a plurality of different health screening measures that can be displayed to a user utilizing machine learning or a decision tree model. In this way, the user can be provided with a plurality of questions during the first set of questions such that the user is performing a plurality of health screening measures over the course of the first set of questions. For example, the first set of questions can include questions from a plurality of different health screening measures to identify potential conditions of the user based on the responses to the first set of questions.

In some examples, the computing device 570 can include instructions 584 that can be executed by a processor resource 572 to capture a first user response to the first set of questions. As described herein, the first set of questions can be provided to a user through a user interface associated with the mental health platform. In this way, the user interface can be utilized to receive responses from the user. In some examples, the first set of questions are multiple-choice questions from the plurality of different health screening measures. The different multiple-choice responses can correspond to different scoring associated with a corresponding health screening measure to indicate a likelihood of a particular health condition of the user. In some examples, the multiple health screening measures can be based on a cumulative score of a plurality of questions to identify if the user has a corresponding condition. For this reason, a plurality of questions from each of a plurality of health screening measures may be provided in order to reach a cumulative score for a particular condition. As described herein, the questions provided by the mental health platform can be based on answers to previous questions such that a greater number of questions are provided to the user for conditions that are identified as more likely based on the previous answers. In this way, the patient responds to relatively more questions associated with conditions that the particular patient is more likely to have based on previous responses or patient entered data.

In some examples, the computing device 570 can include instructions 586 that can be executed by a processor resource 572 to generate a score for a health screen associated with the first user response. As described herein, the score for the health screen can be based on a score associated with each of the multiple-choice questions associated with each of the plurality of health screening measures. In some examples, the score for the health screen includes a cumulative score for answers of the first user for each of the corresponding plurality of health screening measures. In this way, the score can be utilized to identify which of the conditions corresponding to the plurality of health screening measures the first user may have or may be provided with additional questions to determine whether the first user has a particular condition.

In some examples, the computing device 570 can include instructions 588 that can be executed by a processor resource 572 to generate a second set of questions based on the first user response to the first set of questions and the score for the health screen. In some examples, the score and/or responses to the first set of questions are utilized to generate a second set of questions. In some examples, the second set of questions can be generated to elicit responses associated with diagnosing conditions that were identified as possible conditions for the first user based on the first set of questions. In some examples, the second set of questions can be dynamically generated such that a question is generated based on a previous response to a previous question. For example, a first question can be generated based on the first set of questions. In this example, a second question can be generated based on an answer to the first question and a third question can be generated based on an answer to the second question. In this way, the subsequent questions of the second set of questions can be based on previous responses or a plurality of previous responses.

The second set of questions can include a variety of question types. For example, the second set of questions an include multiple choice questions, short answer questions, and/or essay questions, among other types of questions that require different types of answers. In these examples, the answers to the second set of questions can be recorded utilizing a video recorder and/or an audio recorder. For example, the answers to the second set of questions can be captured through a video camera and microphone to collect video data and audio data associated with the answer.

In some examples, the computing device 570 can include instructions 590 that can be executed by a processor resource 572 to capture video and audio data of the user during a second user response to the second set of questions. As described herein, the video data and audio data is collected to identify digital biomarkers. In some examples, the digital biomarkers are utilized to quantify an emotional reaction or add qualitative information to the answer provided by the user. In some examples, the user is presented with a question of the second set of questions. In these examples, the user can select to record an answer through the user interface and record the response to the question. The audio and video data that is recorded during the response to the question is provided to the mental health platform to be analyzed as described herein.

In some examples, the computing device 570 includes instructions to generate an audio baseline for the user during a third user response to a script, extract a feature from the audio data of the user during the second user response, and generate a feature score for the feature based on the generated audio baseline. In these examples, the feature score is utilized to generate a third set of questions. As described herein, the script is a generated question or instructions to say a particular sentence or phrase to capture a vocal pattern or visual pattern of the user for the intake session. For example, the script can include an instruction to count from one to ten.

In these examples, the computing device 570 can include instructions to generate a plurality of additional audio baselines for the user over a period of time to generate a model of vocal patterns of the user. In these examples, the model of vocal patterns includes a measure of a plurality of vocal properties of the user over the period of time. In some examples, the computing device 570 includes instructions to compare video and audio data of the user during a supervised response to video and audio data of the user during a non-supervised response. As described herein, the supervised response is a response provided when a guardian of the user is present, and a non-supervised response is a response provided when the guardian of the user is not present.

In these examples, the computing device 570 includes instructions to generate the third set of questions based on the comparison between the supervised response and the non-supervised response. In some examples, the third set of questions can be generated based on whether the patient is in a supervised situation or an unsupervised situation. For example, the mental health platform can generate a first set of questions when the user or patient is under supervised conditions and a second set of questions when the user or patient is under non-supervised conditions. This can be initiated by the mental health platform based on differences between data collected during supervised responses and non-supervised responses.

In some examples, the audio and video data is utilized to extract features that can be categorized. The extracted features can be categorized to determine a particular score of the extracted features that can be utilized to determine whether the user has a particular condition. In other examples, the extracted features are utilized to extract information of the answer and a subsequent question can be generated based on the extracted information of the answer and/or score of the extracted feature. In this way, the mental health platform can simultaneously generate a score of the answers to the second set of questions even when the second set of questions include non-multiple-choice questions. In addition, the mental health platform can utilize the short answer or essay answers to the non-multiple-choice questions to generate a subsequent question to the user.

In some examples, the computing device 570 can include instructions 592 that can be executed by a processor resource 572 to generate an electronic health record (EHR) for the user that includes data captured during the intake session and a recommended clinical pathway for the user. As used herein, an electronic health record includes a health record that is stored in an electronic database.

In some examples, the electronic health record includes a plurality of information related to a plurality of different patients and the patients corresponding recommended clinical pathway. For example, a particular patient can include patient entered health information, the answers and scores of the evidence based system measures, the digital biomarkers received during intake sessions, digital biomarker base lines and/or other information related to the particular patient. In this example, the electronic health record includes the recommended clinical pathway for the particular patient. As described herein, the recommended clinical pathway includes a plurality of steps to be performed to provide a medical service to the particular patient based on an identified set of medical conditions.

In some examples, the computing device 570 can include instructions to compare the clinical pathway for the user to a plurality of other clinical pathways associated with other users that include corresponding entered health data. In these examples, the generated clinical pathway for a particular user is compared to a corresponding clinical pathway for a plurality of other users that include similar medical data (e.g., age, gender, combination of conditions, etc.). In these examples, the corresponding treatment trajectory predictions and/or results of particular treatments can be compared to generate updates of the clinical pathway recommendation. The updates of the clinical pathway recommendation can include additional time for particular steps, additional coaching associated with the process, among other updates that can be based on the success rates associated with the plurality of other users.

The recommended clinical pathway can include a generated list of the plurality of tasks. In some examples, a first task needs to be completed before starting or completing a second task. In these examples, the recommended clinical pathway generates a notification to a user that is to perform the corresponding first task. In these examples, the user to perform the first task can be provided with a notification when the user is to perform or being performing the task. When the user completes the task, the user is able to utilize the user interface of the mental health platform to indicate that the task has been completed. The mental health platform generates a notification to a different user that the task has been completed when the different user is to perform a subsequent task. In this way, the mental health platform is able to allow a plurality of remote health professionals to perform or execute the recommended clinical pathway for a plurality of patients that utilize the mental health platform.

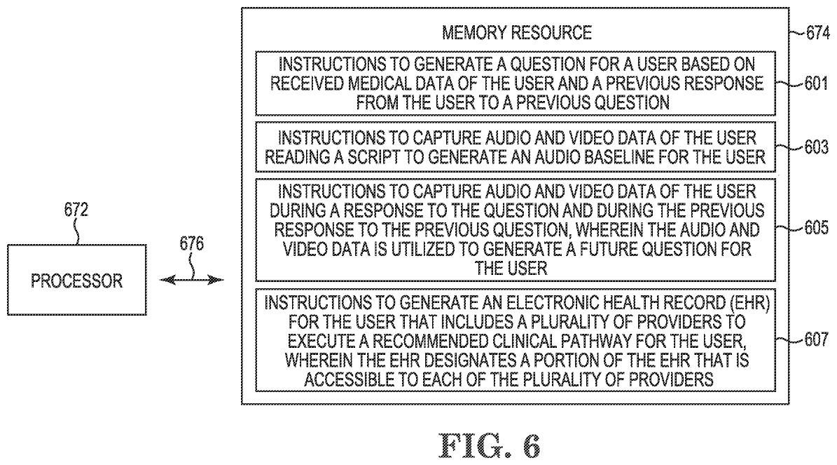

FIG. 6 illustrates an example of a memory resource 674 storing instructions for executing a mental health platform. In some examples, the memory resource 674 can be a part of a computing device or controller that can be communicatively coupled to a computing system that includes image capture devices. For example, the memory resource 676 can be part of a computing device 570 as referenced in FIG. 5 and communicatively coupled to a plurality of devices (e.g., remote computing devices, remote display devices, etc.). In some examples, the memory resource 674 can be communicatively coupled to a processor resource 672 that can execute instructions 601, 603, 605, 607 stored on the memory resource 674. For example, the memory resource 674 can be communicatively coupled to the processor resource 672 through a communication path 676. In some examples, a communication path 676 can include a wired or wireless connection that can allow communication between devices and/or components within a single device.

The memory resource 674 may be electronic, magnetic, optical, or other physical storage device that stores executable instructions. Thus, a non-transitory machine readable medium (MRM) (e.g., a memory resource 674) may be, for example, a non-transitory MRM comprising Random-Access Memory (RAM), read-only memory (ROM), an Electrically-Erasable Programmable ROM (EEPROM), a storage drive, an optical disc, and the like. The non-transitory machine readable medium (e.g., a memory resource 674) may be disposed within a controller and/or computing device. In this example, the executable instructions 601, 603, 605, 607 can be “installed” on the device. Additionally, and/or alternatively, the non-transitory machine readable medium (e.g., a memory resource 674) can be a portable, external or remote storage medium, for example, that allows a computing system to download the instructions 601, 603, 605, 607 from the portable/external/remote storage medium. In this situation, the executable instructions may be part of an “installation package”. As described herein, the non-transitory machine readable medium (e.g., a memory resource 674) can be encoded with executable instructions for executing a mental health platform.

The instructions 601, when executed by the processor resource 672, can include instructions to generate a question for a user based on received medical data of the user and a previous response from the user to a previous question. As described herein, the mental health platform utilizes patient entered medical data to generate or identify seed questions to provide to a particular user. In some examples, the mental health platform generates a questions based on a previous question, each of a plurality of previous of questions, and/or the patient entered medical data. In this way, the mental health platform dynamically generates a plurality of questions that are subsequentially directed to particular conditions that are most likely to be present within a particular patient.

The instructions 603, when executed by the processor resource 672, can include instructions to capture audio and video data of the user reading a script to generate an audio baseline for the user. As described herein, the script can be a list of numbers or basic questions that allow a baseline to be generated for a particular patient. For example, the user can be instructed to count to a particular number (e.g., 10, 20, etc.). The baseline for the audio or video data of the user can be generated to identify properties of the audio or video data when the user is not under additional stress of a particular question. In this way, the baseline for the user can be utilized to identify when particular questions or situations have a greater or lesser affect on the patient.

In some examples, the reading of the script is performed prior to each of a plurality of intake sessions over a period of time. In this way, a baseline for a first session at a first time can be compared to a baseline for a second session at a second time. The baseline for the user can be monitored over a period of time to determine changes in the baseline of the user. In other examples, the baseline for a particular start of an intake session can be utilized for that particular intake session. For example, the user may have experienced an event prior to a particular intake session which may affect a property of the audio data and/or video data. For example, the pitch, tone, or clarity of a patient's voice may be altered or affected when the patient manifests symptoms. In this way, a baseline of the audio data and video data for a user can be captured prior to providing questions to the user or before providing questions that are answered through audio or video data.

The instructions 605, when executed by the processor resource 672, can include instructions to capture audio and video data of the user during a response to the question and during the previous response to the previous question. In these examples, the audio and video data is utilized to generate a future question for the user. In some examples, the instructions to capture audio and video data includes receiving the audio and video data from a computing device of the user. In these examples, the user may capture the audio and video data in response to the question and upload the captured audio and video data to the mental health platform.

The mental health platform receives the audio and video data to analyze the data as described herein. For example, the audio and video data can be compared to the baseline to identify differences between the properties of the audio and video data of the answer and the baseline. In another example, the features of the audio and video can be extracted and scored to generate a score of non-multiple choice answers to determine if the patient has a particular condition. In addition, the audio and video data can be utilized to categorize the extracted features of the audio and video data to generate a subsequent question.

The instructions 607, when executed by the processor resource 672, can include instructions to generate an electronic health record (EHR) for the user that includes a plurality of providers to execute a recommended clinical pathway for the user. In some examples, the memory resource 674 can include instructions to generate a plurality of tasks to be executed by the plurality of providers for the recommended clinical pathway for the user. For example, the electronic health record for the user can include a plurality of tasks that are each assigned to a particular provider of the plurality of providers. In this example, a provider can send a notification to the mental health platform that the task is complete and a different provider can be notified by the mental health platform that their task is ready to be complete. In another example, the mental health platform can receive an indication from a first provider of the plurality of providers that a first task of the plurality of tasks has been completed and generate a notification to a second provider of the plurality of providers that the first task has been completed and a second task associated with the second provider is to be completed.

In some examples, the recommended clinical pathway includes a plurality of workflows that each include a corresponding plurality of providers to perform tasks associated with a corresponding workflow. For example, a each of the plurality of providers have a plurality of tasks that are to be completed in a particular order. In some examples, the particular order includes tasks that need to be completed by other providers. In this way, the mental health platform generates the notifications to the other users when a particular task is completed. In some examples, the plurality of workflows are generated with a sequence of completion based on due dates and each task of the plurality of workflows are initiated upon completion of a previous task of the sequence. In some examples, the computing device 770 can generate a user interface that is displayed on the display device 778. The user interface can include the standardized workflow of sequential processes for the recommended clinical pathway for the user. In these examples, the user interface allows a first provider of the plurality of providers to mark a first process as complete before allowing a second provider to mark a second process as complete.

In these examples, the electronic health record designates a portion of the electronic health record that is accessible to each of the plurality of providers. As described herein, the electronic health record can include a plurality of tasks to execute a treatment plan for a particular user or particular patient. In some examples, the electronic health record includes a plurality of tasks that are to be completed by a plurality of different medical and non-medical professionals. In some examples, the mental health platform can collect audio and video data for a plurality of questions during the recommended clinical pathway and generate a treatment trajectory probability curve based on the collected audio and video data. In this way, the data from the electronic health record is utilized to generate a treatment trajectory probability curve for the user and update the recommended clinical pathway for the user. For example, the mental health platform can generate an updated clinical pathway based on the treatment trajectory probability curve.

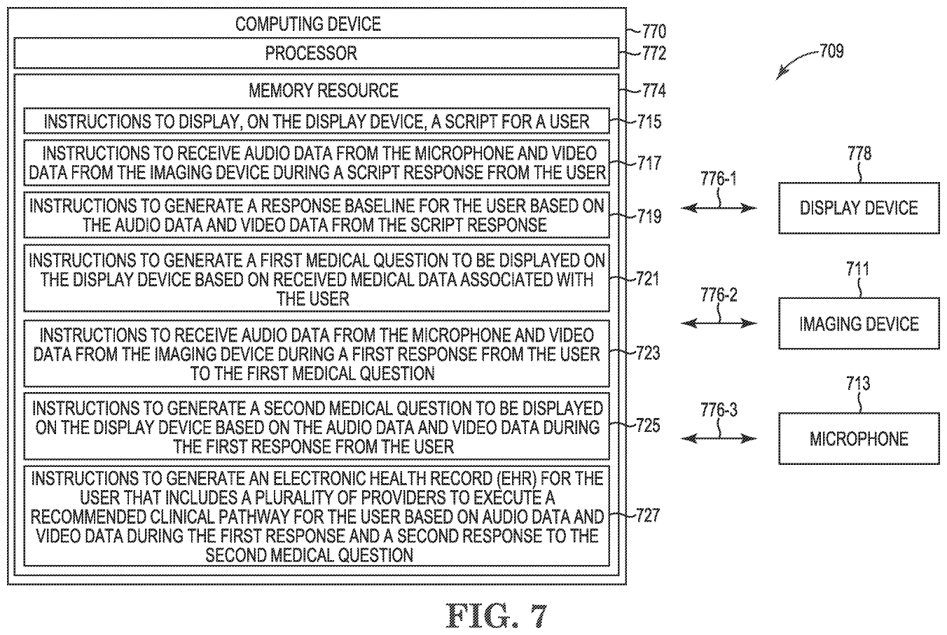

FIG. 7 illustrates an example of a system 709 including a computing device 770 for executing a mental health platform. In some examples the computing device 770 can be a device that includes a processor resource 772 communicatively coupled to a memory resource 774. As described herein, the memory resource 774 can include or store instructions 715, 717, 719, 721, 723, 725, 727, that can be executed by the processor resource 772 to perform particular functions.

The system 709 includes a display device 778, imaging device 711, and microphone 713. In some examples, the display device 778, imaging device 711, and/or microphone 713 can be associated with a user device or patient device. In this example, the user device can be a computing device that is collecting the patient entered data, biometric data, and/or evidence based system measures. In these examples, the computing device 770 can collect the patient entered data, biometric data, and/or evidence based system measures through communication paths 776-1, 776-2, 776-3. In this way the computing device 770 can be a local device associated with the display device 778, imaging device 711, and microphone 713 or a remote device communicatively coupled through a network connection.

In some examples, the computing device 770 can include instructions 715 that can be executed by a processor resource 772 to display, on the display device 778, a script for a user. As described herein, a script can include an instruction to recite a particular phrase or group of words in order to generate a baseline of audio properties for the user. In some examples, the script is an instruction to count to ten. In these examples, the script can be designed to elicit a response from a user that is void of emotion or void of a reactionary response. In this way, the user's emotion or reactionary response to a particular question can be more easily identified through a change in the audio properties of the captured audio data during an answer to a particular question.

In some examples, the computing device 770 can include instructions 717 that can be executed by a processor resource 772 to receive audio data from the microphone 713 and video data from the imaging device 711 during a script response from the user. As described herein, the user can be instructed to read the script when the microphone 713 is recording audio data and the imaging device 711 is capturing video data. In this way, the audio properties and video properties can be utilized to generate a baseline for the user.

In some examples, the computing device 770 can include instructions 719 that can be executed by a processor resource 772 to generate a response baseline for the user based on the audio data and video data from the script response. As described herein, a response baseline for the user can include a model of the user's vocal patterns and/or visual patterns while reading a script to eliminate emotional influence from the baseline. In some examples, the response baseline can be generated at a beginning of each intake session or interaction with the mental health platform over a period of time. In this way, a baseline can be generated and utilized during the intake session. In addition, the baseline for a particular user can be monitored over time to identify changes in the vocal patterns and/or visual patterns of a particular user. In these examples, the computing device 770 can include instructions to compare the response baseline to a plurality of collected audio and video data of responses from the user over a period of time to identify changes in acoustic features of the user over time.

In some examples, the computing device 770 can include instructions 721 that can be executed by a processor resource 772 to generate a first medical question to be displayed on the display device 778 based on received medical data associated with the user. In some examples, the first medical question is a multiple choice question from one of a plurality of different condition screening measures. As described herein, the condition screening measures can be standardized questionnaires that include questions and multiple choice answers where each answer corresponds to a particular scoring value. In this way, a cumulative score from the answers to a plurality of questions can be utilized to determine if a user has a corresponding condition associated with the condition screening measures.

In some examples, the first medical question can be a question that was generated based on a previous response to a previous medical question. In these examples, the audio data of the previous response can be utilized to extract particular features of the audio data and determine an answer and/or vocal patterns of the user during the recorded answer to the previous question. In these examples, the first medical question can be based on the answer and/or vocal patterns of the user that may indicate a particular condition and/or a need for a particular follow-up question.

In some examples, the computing device 779 can include instructions 723 that can be executed by a processor resource 772 to receive audio data from the microphone 713 and video data from the imaging device 711 during a first response from the user to the first medical question. As described herein, the audio data and the video data that is captured from the response to the first medical question can be analyzed based on the word selection, vocal patterns, and/or baseline for the user. In this way, the properties of the audio data and video data can be utilized to score the response to the first medical question and the score can be utilized to determine a condition of the user utilizing the answers that can be non-multiple choice questions. In addition, the audio data and video data can be utilized to generate a subsequent question for the user.

In some examples, the computing device 770 can include instructions 725 that can be executed by a processor resource 772 to generate a second medical question to be displayed on the display device 778 based on the audio data and video data during the first response from the user. As described herein, the second medical question can be a follow-up question based on the response to the first medical question. In these examples, the answer to the first medical question can include features the can indicate a particular condition of the user and the second medical question can be generated to further analyze or generate a score of the particular condition.

In some examples, the computing device 770 can include instructions 727 that can be executed by a processor resource 772 to generate an electronic health record (EHR) for the user that includes a plurality of providers to execute a recommended clinical pathway for the user based on audio data and video data during the first response and a second response to the second medical question. The electronic health record is a set of stored data that can include medical data related to the user, historical medical records of the user, a clinical pathway recommendation for the user, a treatment trajectory prediction for the user, and/or results of the intake session for the user. In some examples, the computing device 770 includes instructions to select the recommended clinical pathway from a plurality of clinical pathways that each include a standardized workflow of sequential processes. In these examples, the sequential processes or tasks can be provided by different medical and non-medical professionals.

In some examples, the computing device 770 can include instructions to generate a medical condition and condition severity of the user based on the audio data and video data during the first response and the second response of the user. As described herein, the medical condition and condition severity can be based on the score associated with the health condition screening questions and dynamically generated questions. In this way, the medical condition and condition severity for a particular intake session can be stored in the electronic health record for the patient.

In some examples, the computing device 770 can select the plurality of providers based on the medical condition and condition severity of the user. For example, a particular condition can include a treatment plan that utilizes a particular type of medical professional. In other examples, the condition severity can include a different treatment plan that utilizes a particular type of medical professional with a support team to ensure the user is able to complete the treatment plan at the particular condition severity.

In the foregoing detailed description of the disclosure, reference is made to the accompanying drawings that form a part hereof, and in which is shown by way of illustration how examples of the disclosure may be practiced. These examples are described in sufficient detail to enable those of ordinary skill in the art to practice the examples of this disclosure, and it is to be understood that other examples may be utilized and that process, electrical, and/or structural changes may be made without departing from the scope of the disclosure. Further, as used herein, “a” refers to one such thing or more than one such thing.

The figures herein follow a numbering convention in which the first digit corresponds to the drawing figure number and the remaining digits identify an element or component in the drawing. For example, reference numeral 102 may refer to element 102 in FIG. 1 and an analogous element may be identified by reference numeral 302 in FIG. 3. Elements shown in the various figures herein can be added, exchanged, and/or eliminated to provide additional examples of the disclosure. In addition, the proportion and the relative scale of the elements provided in the figures are intended to illustrate the examples of the disclosure and should not be taken in a limiting sense.

It can be understood that when an element is referred to as being “on,” “connected to”, “coupled to”, or “coupled with” another element, it can be directly on, connected, or coupled with the other element or intervening elements may be present. In contrast, when an object is “directly coupled to” or “directly coupled with” another element it is understood that are no intervening elements (adhesives, screws, other elements) etc.

The above specification, examples, and data provide a description of the system and method of the disclosure. Since many examples can be made without departing from the spirit and scope of the system and method of the disclosure, this specification merely sets forth some of the many possible example configurations and implementations.

What is claimed is:

1. A computing device, comprising:
a processor resource; and
a non-transitory memory resource storing machine-readable instructions stored thereon that, when executed, cause the processor resource to:
initiate an intake session for a user;
generate a first set of questions based on entered health data related to the user;
capture a first user response to the first set of questions;
generate a score for a health screen associated with the first user response;
generate a second set of questions based on the first user response to the first set of questions and the score for the health screen;
capture video and audio data of the user during a second user response to the second set of questions; and
generate an electronic health record (EHR) for the user that includes data captured during the intake session and a recommended clinical pathway for the user based on the score for the health screen and video and audio data captured during second set of questions.

2. The computing device of claim 1, wherein the processor resource is to:
generate an audio baseline for the user during a third user response to a script;
extract a feature from the audio data of the user during the second user response; and
generate a feature score for the feature based on the generated audio baseline, wherein the feature score is utilized to generate a third set of questions.

3. The computing device of claim 2, wherein the processor resource is to generate a plurality of additional audio baselines for the user over a period of time to generate a model of vocal patterns of the user, wherein the model of vocal patterns includes a measure of a plurality of vocal properties of the user over the period of time.

4. The computing device of claim 1, wherein the processor resource is to compare video and audio data of the user during a supervised response to video and audio data of the user during a non-supervised response, wherein the supervised response is a response provided when a guardian of the user is present, and a non-supervised response is a response provided when the guardian of the user is not present.

5. The computing device of claim 4, wherein the processor resource is to generate a third set of questions based on the comparison between the supervised response and the non-supervised response.

6. The computing device of claim 1, wherein the processor resource is to determine a lexical complexity of the user based on the second user response.

7. The computing device of claim 1, wherein the processor resource is to compare the recommended clinical pathway for the user to a plurality of other clinical pathways associated with other users that include corresponding entered health data.

8. A non-transitory memory resource storing machine-readable instructions stored thereon that, when executed, cause a processor resource to:
generate a question for a user based on received medical data of the user and a previous response from the user to a previous question;
capture audio and video data of the user reading a script to generate an audio baseline for the user;
capture audio and video data of the user during a response to the question and during the previous response to the previous question, wherein the audio and video data is utilized to generate a future question for the user; and
generate an electronic health record (EHR) for the user that includes a plurality of providers to execute a recommended clinical pathway for the user, wherein the EHR designates a portion of the EHR that is accessible to each of the plurality of providers.

9. The memory resource of claim 8, wherein the processor resource is to generate a plurality of tasks to be executed by the plurality of providers for the recommended clinical pathway for the user.

10. The memory resource of claim 8, wherein the processor resource is to:
receive an indication from a first provider of the plurality of providers that a first task of the plurality of tasks has been completed; and
generate a notification to a second provider of the plurality of providers that the first task has been completed and a second task associated with the second provider is to be completed.

11. The memory resource of claim 8, wherein the processor resource is to:
collect audio and video data for a plurality of questions during the recommended clinical pathway; and
generate a treatment trajectory probability curve based on the collected audio and video data.

12. The memory resource of claim 11, wherein the processor resource is to generate an updated clinical pathway based on the treatment trajectory probability curve.

13. The memory resource of claim 8, wherein the recommended clinical pathway includes a plurality of workflows that each include a corresponding plurality of providers to perform tasks associated with a corresponding workflow.

14. The memory resource of claim 13, wherein the plurality of workflows are generated with a sequence of completion based on due dates and each task of the plurality of workflows are initiated upon completion of a previous task of the sequence.

15. A system, comprising:
a display device;
an imaging device;
a microphone; and
a processor to:
display, on the display device, a script for a user;
receive audio data from the microphone and video data from the imaging device during a script response from the user;
generate a response baseline for the user based on the audio data and video data from the script response;
generate a first medical question to be displayed on the display device based on received medical data associated with the user;
receive audio data from the microphone and video data from the imaging device during a first response from the user to the first medical question;
generate a second medical question to be displayed on the display device based on the audio data and video data during the first response from the user; and
generate an electronic health record (EHR) for the user that includes a plurality of providers to execute a recommended clinical pathway for the user based on audio data and video data during the first response and a second response to the second medical question, wherein the EHR designates a portion of the EHR that is accessible to each of the plurality of providers.

16. The system of claim 15, wherein the processor is to generate a medical condition and condition severity of the user based on the audio data and video data during the first response and the second response of the user.

17. The system of claim 16, wherein the processor is to select the plurality of providers based on the medical condition and condition severity of the user.

18. The system of claim 15, wherein the processor is to select the recommended clinical pathway from a plurality of clinical pathways that each include a standardized workflow of sequential processes.

19. The system of claim 18, wherein the processor is to generate a user interface that includes the standardized workflow of sequential processes for the recommended clinical pathway for the user, wherein the user interface allows a first provider of the plurality of providers to mark a first process as complete before allowing a second provider to mark a second process as complete.

20. The system of claim 15, wherein the processor is to compare the response baseline to a plurality of collected audio and video data of responses from the user over a period of time to identify changes in acoustic features of the user over time.
